(19) United States
(12) Patent Application Publication
(10) Pub. No.: US 2006/0041227 A1
(43) Pub. Date: Feb. 23, 2006

(54) Devices for blocking spaces defined within hollow structures

(75) Inventor: Yoshihiro Takabatake, Toyota-shi (JP)

Correspondence Address: CONLEY ROSE, P.C., P. O. BOX 3267, HOUSTON, TX, 77253-3267, US

(73) Assignees: Kyowa Sangyo Co., LTD., Toyota-shi (JP); Shinwa Seiko Co., LTD., Toyota-shi (JP)

(21) Appl. No.: 11/208,957

(22) Filed: Aug. 22, 2005

(30) Foreign Application Priority Data
Aug. 23, 2004 (JP) 2004-242281

(51) Int. Cl. A61M 29/00

(52) U.S. Cl. 604/99.04

(57) ABSTRACT

A blocking device for mounting within a space defined within a hollow structure includes a blocking member and a fixing device. The blocking member may be disposed on one side of a wall portion extending within the space. The blocking member has an expandable member and a holder for holding the expandable member. The expandable member is expandable by heat to form a foam member. The fixing device has a fixing member that may be disposed on the other side of the wall portion within the space. The fixing device also has a connecting member that may extend through an insertion hole formed in the wall portion. The connecting member connects the blocking member to the fixing member.

This application claims priority to Japanese patent application serial number 2004-242281, the contents of which are incorporated herein by reference.

### BACKGROUND OF THE INVENTION

1. Field of the Invention

The present invention relates to devices for blocking spaces defined within hollow structures. In particular to blocking devices that include expandable members expanding by external heat to form foam members for blocking the spaces.

2. Description of the Related Art

For example, Japanese Laid-Open Patent Publication No. 2001-79845 teaches a device for blocking a space defined within a hollow structure, such as a hollow panel of an automobile body (e.g., a pillar, a rocker panel, a roof side panel, or a wheel housing), by a foam member in order to improve the damping and sound insulating properties of the hollow structure.

The device disclosed in the above publication has a blocking member constituted by an expandable member that is expanded by external heat to form a foam member and a holder plate for holding the expandable member within the space of the hollow structure. The blocking member is mounted within the space by means of clips that are made of synthetic resin and are integrated with the holder plate.

In the case in which the hollow structure is a hollow panel, the hollow panel may be dipped into a bath containing a liquid coating material for forming electro-deposition coatings on both the internal and external surfaces of the hollow panel. After the internal and external surfaces are coated with the electro-deposition coatings, the hollow panel may be conveyed into a heating furnace where the coatings are baked. The expandable member retained by the holder plate may then expand to form a foam member as a result of the heat applied during the baking process, so that the foam member blocks the space within the hollow structure.

However, according to the blocking device of the above publication, the blocking member of the blocking device is mounted to the wall surface of the hollow structure by means of clips made of synthetic resin. Therefore, there has been a problem in that the mounting strength of the blocking member to the wall of the hollow structure is not sufficient. Thus, in the event that the coating material for electro-deposition surges into the space of the hollow structure, there is a possibility that the clips are forcibly removed from the corresponding mounting holes formed in the wall of the hollow structure. In some cases, the blocking member of the blocking device may fail (e.g., breaking of mounting structures, or being moved to an incorrect position or orientation) due to the pressure of the flow of the coating material. When this occurs, the expandable member may expand in an unintended direction. Consequently, it may be impossible to reliably block the space with the expanded foam member so as to not result in any significant clearance between the blocking device and the walls of the hollow structure.

### SUMMARY OF THE INVENTION

It is accordingly an object of the present invention to teach improved techniques for reliably fixing the bodies of the blocking devices in position within a space of a hollow structure.

In one aspect of the present teachings, blocking devices for mounting within a space defined within a hollow structure are taught. The hollow structure may have a wall portion extending within the space. The blocking device may include a blocking member and a fixing device. The blocking member may be disposed on one side of the wall portion within the space. The blocking member may include an expandable member and a holder for holding the expandable member. The expandable member may be expandable by heat to form a foam member. The fixing device may include a fixing member that may be disposed on the other side of the wall portion within the space. The fixing device further may include a connecting member(s) that may extend through an insertion hole(s) formed in the wall portion. The connecting member(s) connects the blocking member and the fixing member.

With this arrangement, the blocking member may be fixed in position by the fixing member via the connecting member that extends through the insertion hole formed in the wall portion of the hollow structure. Therefore, the blocking member can be reliably fixed in position in comparison with the known fixing device utilizing clips. In addition, the blocking member may be reliably prevented from failing within the space of the hollow structure, even in the event that a liquid coating material for electro-deposition surges into the space of the hollow structure.

In one embodiment, the expandable member has a first side and a second side opposite to each other. The holder includes a first holder plate and a second holder plate respectively disposed on the first side and the second side of the expandable member. The first holder plate has an engaging member(s). The second holder plate has an engaging hole(s) positioned to correspond to the engaging member(s). The connecting member has a latching hole, so that the first engaging member is engageable with the engaging hole while the engaging member extends through the latching hole.

With this arrangement, the engaging member for assembling the first holder plate to the second holder plate can also be used as a latching member that may prevent the connecting member from being removed from the blocking member. Therefore, the manufacturing cost of the blocking device can be reduced.

In another embodiment, an engaging claw is formed integrally with a circumferential edge of the engaging hole in order to prevent the engaging member from being accidentally removed from the engaging hole. Therefore, it is possible to reliably prevent the first and second holder plates from being accidentally removed from each other. In other words, it is possible to prevent the expandable member from expanding at an unintended position or orientation within the space.

In a further embodiment, the connecting member(s) may have an engaging member(s) disposed on an end portion(s) of the connecting member(s). The blocking member may have an engaging hole(s) for engagement with the engaging member(s). The engaging hole may have an engaging portion formed on an inner wall of the engaging hole and may be engageable with the engaging member for preventing the engaging member from being accidentally removed from the engaging hole. Therefore, it is possible to easily prevent the connecting member from being accidentally removed from the blocking device. The engaging member(s) may be formed integrally with the holder and the connecting member(s) may be formed integrally with the fixing member.

In a further embodiment, the holder plate has at least one rib for preventing the blocking member from falling down within the space of the hollow structure. Therefore, even in the event that a liquid coating material for electro-deposition surges into the space of the hollow structure, the ribs may reliably hold the blocking member in an upright position.

In a further embodiment, the fixing member may have a second expandable member and a second holder for holding the second expandable member. The expandable member is expandable by heat so as to form a second foam member. With this arrangement, the fixing member can also serve as a second blocking member. Therefore, the fixing member can further reliably fix the blocking member in position by the fixing member. In addition, it is possible to simultaneously block both sides of the wall portion within the space. This arrangement is particularly advantageous if the wall portion is a partition wall extending within the space in the longitudinal direction of the hollow structure in order to separate the space into a first space and a second space. Thus, the blocking member may be disposed within the first space to block the first space and the fixing member may be disposed within the second space to block the second space.

### BRIEF DESCRIPTION OF THE DRAWINGS

FIG. 1 is an exploded perspective view of a representative blocking device for blocking a space within a hollow structure; and

FIG. 2 is an exploded front view of the blocking device; and

FIG. 3 an exploded perspective view of a blocking member of the blocking device; and

FIG. 4 is a cross-sectional view taken along line IV-IV in FIG. 2; and

FIG. 5 is a front view of the blocking device in an assembled state within the space of the hollow structure; and

FIG. 6 is a cross-sectional view taken along line VI-VI in FIG. 5; and

FIG. 7 is a cross-sectional view similar to FIG. 6 but showing foam members produced as a result of expansion by external heat of the expandable members of the blocking device; and

FIGS. 8(a) is a plan view of an end portion of a connecting member having an engaging member according to a first modification of an engaging device between the connecting member and a blocking body; and

FIG. 8(b) is a sectional view taken along line VIII-VIII in FIG. 8(a) and showing the state where the engaging member engages with an engaging hole formed in the blocking body; and

FIG. 9(a) is a front view of an end portion of a connecting member having engaging members according to a second modification; and

FIG. 9(b) is a front view of the end portion of the connecting member and showing the state where the engaging members engages with an engaging hole formed in the blocking body; and

FIG. 10(a) is a front view of an end portion of a connecting member having engaging member according to a third modification; and

FIG. 10(b) is a front view of the end portion of the connecting member and showing the state where the engaging member engages with an engaging hole formed in the blocking body.

### DETAILED DESCRIPTION OF THE INVENTION

Each of the additional features and teachings disclosed above and below may be utilized separately or in conjunction with other features and teachings to provide improved blocking devices for hollow structures and methods of manufacturing such blocking devices. Representative examples of the present invention, which examples utilize many of these additional features and teachings both separately and in conjunction with one another, will now be described in detail with reference to the attached drawings. This detailed description is merely intended to teach a person of skill in the art further details for practicing preferred aspects of the present teachings and is not intended to limit the scope of the invention. Only the claims define the scope of the claimed invention. Therefore, combinations of features and steps disclosed in the following detailed description may not be necessary to practice the invention in the broadest sense, and are instead taught merely to particularly describe representative examples of the invention. Moreover, various features of the representative examples and the dependent claims may be combined in ways that are not specifically enumerated in order to provide additional useful embodiments of the present teachings.

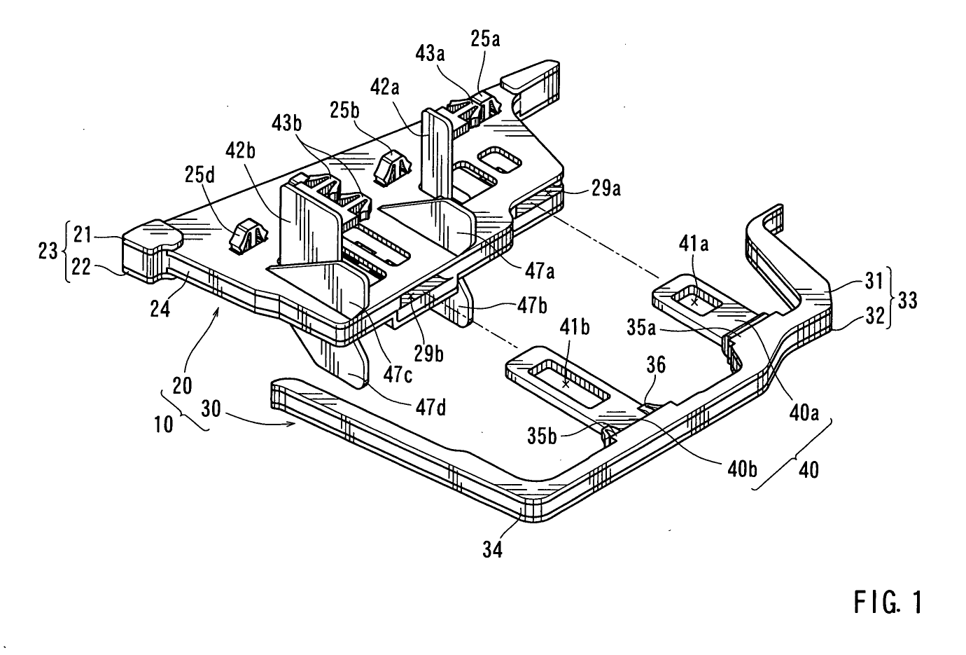

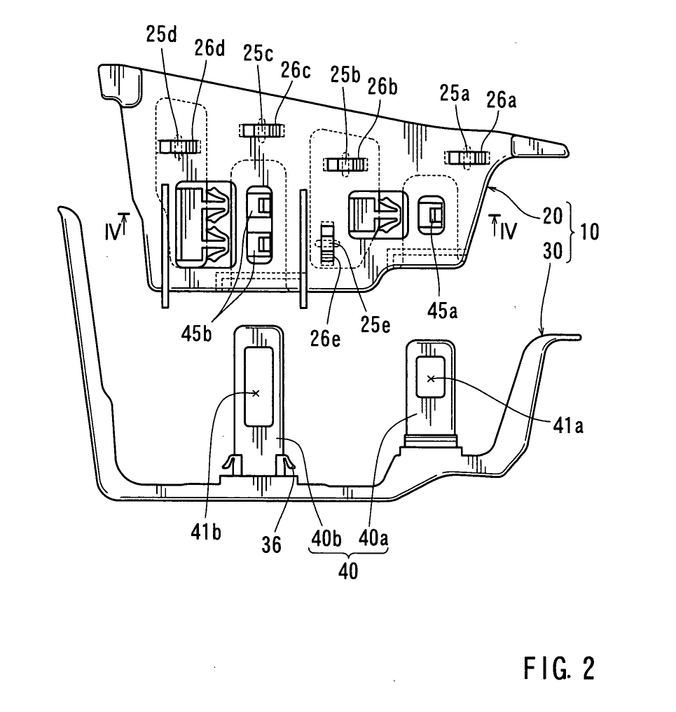

A representative embodiment will now be described with reference to FIGS. 1 to 7. Referring to FIGS. 1 and 2, a representative blocking device 10 generally includes a blocking member 20, a fixing member 30, and a connecting device 40 used for connecting the blocking member 20 to the fixing member 30.

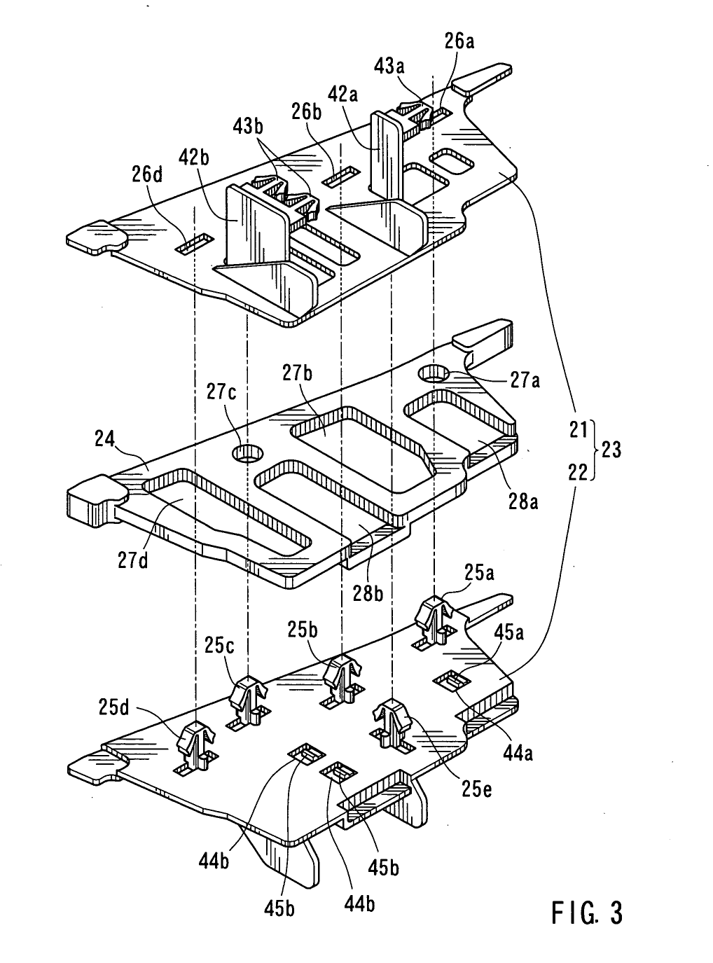

The construction of the blocking member 20 will be described first. Referring to FIG. 3, the blocking member 20 has a holder 23 and an expandable member 24. The expandable member 24 is held by the holder 23 within the space of a hollow structure, as will be explained later. The holder 23 includes a pair of a first holder plate 21 and a second holder plate 22, which may preferably be made of heat-resisting thermoplastic resin, such as 66-nylon, and molded by an injection molding process. More specifically, the first and second holder plates 21 and 22 are configured to substantially conforming to the cross-section of the space within the hollow structure.

As shown in FIG. 3, five retaining clips 25a to 25e are integrally formed with the second holder plate 22 and extend from one side surface of the second holder plate 22. The first holder plate 21 is positioned opposing the second holder plate 22 and has five retaining holes 26a to 26e (26c and 26e can not be seen in FIG. 3) respectively corresponding to the retaining clips 25a to 25e. The expandable member 24 is adapted to be positioned between the first and second holder plates 21 and 22 and has four through holes 27a to 27d. Therefore, the retaining clips 25a to 25e may be inserted into the through holes 27a to 27d and then may be engaged with the retaining holes 26a to 26e (retaining clips 25b and 25e both extend into through hole 27b in this example). As a result, the holder plates 21 and 22 may be assembled to each other with the expandable member 24 interleaved therebetween.

In addition, as shown in FIG. 3, two bottomed recesses 28a and 28b are formed in stepped manners in one side surface of the expandable member 24. The insertion holes 29a and 29b are defined between the two bottomed recesses 28a and 28b and a side surface of the first holder plate 21 in order to respectively receive connecting members 40a and 40b of the connecting device 40 (see FIG. 1).

The expandable member 24 may be made of any suitable expandable material. However, the expandable member 24 may preferably be made of synthetic-resin-based expanding material containing an expanding agent mixed therewith for producing foams when externally heated. More preferably, as a main component, the expandable member 24 may have a synthetic resin material having adhesion properties to metal surfaces and/or synthetic resin surfaces. A fibrous material such as glass fibers may be mixed with the synthetic resin material. With this composition, the expanding member 24 may expand to form a foam member when heated during the baking process of the coatings on an automobile body (e.g. at a temperature of about 110° C. to about 190° C.). Such expandable materials having adhesion properties are disclosed, for example, in Japanese Laid-Open Patent Publication No. 9-176356, the contents of which are incorporated herein.

The construction of the fixing member 30 will now be described. The construction of the fixing member 30 is generally the same as the construction of the blocking member 20. Thus, the fixing member 30 has a holder 33 and an expandable member 34 that is held by the holder 33 within the space of a hollow structure. The holder 33 includes a pair of a first holder plate 31 and a second holder plate 32 that may preferably be made of a heat-resisting thermoplastic resin, such as 66-nylon, and molded by an injection molding process. More specifically, the first and second holder plates 31 and 32 are configured to substantially conform to the shape of a circumferential portion of the cross-section of the space within the hollow structure. In this representative embodiment, the first and second holder plates 31 and 32 have substantially elongated U-shaped configurations. The first holder plate 31 and the second holder plate 32 are positioned opposite to each other and are joined together at joint portions 35a and 35b. The connecting members 40a and 40b respectively extend from the joint portions 35a and 35b. The connecting members 40a and 40b have elongated flat plate-shaped configurations and respectively have latching holes 41a and 41b. A clip 36 is integrally formed with the joint portion 35b and is adapted to temporarily engage a corresponding engaging hole formed in the inner wall of the hollow structure.

The constructions of the fixing member 30 and the blocking member 20 will now be described in relation to the connection between the fixing member 30 and the blocking member 20. As shown in FIGS. 1 and 3, a clip 43a is integrally formed with the first holder plate 21 via a hinge 42a. Similarly, two clips 43b are integrally formed with the first holder plate 21 via a hinge 42b. An engaging hole 44a is formed in the second holder plate 22 at a position corresponding to the clip 43a. Similarly, engaging holes 44b are formed in the second holder plate 22 at positions corresponding to the clips 43b. In order to connect the fixing member 30 to the blocking member 20, the connecting members 40a and 40b may first be respectively inserted into the insertion holes 29a and 29b. The clips 43a and 43b may then be respectively engaged with the corresponding engaging holes 44a and 44b. In this state, the clips 43a and 43b respectively extend through the corresponding latching holes 41a and 41b formed in the connecting members 40a and 40b. Therefore, the connecting members 40a and 40b may be prevented from being removed from the insertion holes 29a and 29b. In other words, the connecting members 40a and 40b may be fixedly latched. Consequently, the clips 43a and 43b for assembling the holder plate 21 and 22 together can also be used as latches for preventing the connecting members 40a and 40b from being removed from the blocking member 20. As a result, the manufacturing cost of the blocking device 10 can be reduced.

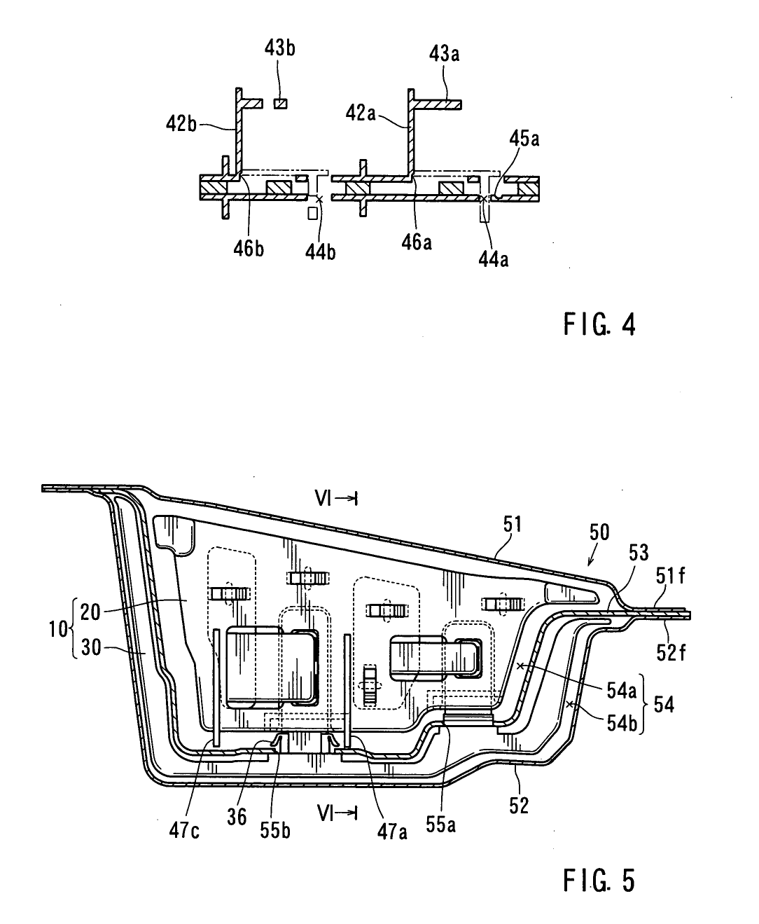

In FIG. 4, chain lines indicate the engaging positions of the clips 43a and 43b with the engaging holes 44a and 44b. Solid lines indicate the disengaging positions of the clips 43a and 43b away from the engaging holes 44a and 44b. As shown in FIGS. 2 to 4, a removal prevention claw 45a may be integrally formed with the circumferential edge of the engaging hole 44a in order to prevent the clip 43a from being inadvertently removed from the engaging hole 44a. Similarly, a removal prevention claw 45b may be integrally formed with the circumferential edge of each of the engaging holes 44b in order to prevent each corresponding clip 43b from being inadvertently removed from its engaging hole 44b. The removal prevention claw 45a (45b) is positioned and configured such that the distal end of the clip 43a (43b) furthest from a pivotal point 46a (46b), which has a flexible reduced thickness portion for the hinge 42a (42b), may contact with the removable prevention claw 45a (45b) so as to cause resilient deformation of the removable prevention claw 45a (45b) when the clip 43a (43b) is moved to a position to engage with the engaging hole 44a (44b). Therefore, a moderate resistance force is applied to the clip 43a (43b) before the clip 43a (43b) completely engages with the engaging hole 44a (44b). Conversely, when the clip 43a (43b) is moved in a removing direction from the engaging hole 44a (44b), the removal prevention claw 45a (45b) may resist against removal of the clip 43a (43b) by resiliently holding the distal end of the clip 43a (43b), the portion most remote from the pivotal point 46a (46b). Therefore, the clip 43a (43b) may be reliably prevented from being removed from the engaging hole 44a (44b). As a result, it is possible to prevent the expandable member 24 from expanding at an unintended position within the space of the hollow structure due to the accidental disassembly or separation of the first and second holders 21 and 22.

The operation of the above first representative embodiment will now be described in relation to the mounting operation of the blocking device 10 within the space of the hollow structure. Referring to FIG. 5, a hollow structure 50 defines a space 54 within which is mounted the blocking device 10. The hollow structure 50 has an inner panel 51 joined to an outer panel 52 at their flange portions 51f and 52f, for example by a spot welding technique, so that the hollow structure 50 may have a tubular configuration. A reinforcement panel 53 is interleaved between the inner panel 51 and the outer panel 52. The reinforcement panel 53 may serve to reinforce the physical strength of the hollow structure 50 and also may serve as a part of the wall of the hollow structure 50. More specifically, the reinforcement panel 53 may serve as a partition wall separating the space 54 into a first space 54a and a second space 54b. The inner panel 51 and the reinforcement panel 53 define the first space 54a. The outer panel 52 and the reinforcement panel 53 define the second space 54b.

As shown in FIG. 5, the blocking member 20 and the fixing member 30 are respectively positioned within the first space 54a and the second space 54b. As a result, the blocking member 20 and the fixing member 30 are positioned on opposite sides with respect to the reinforcement panel 53. Mounting holes 55a and 55b are formed in the reinforcement panel 53. The connecting members 40a and 40b respectively extend through the mounting holes 55a and 55b so as to connect the blocking member 20 together with the fixing member 30. The clip 36 is formed on the joint portion 35b of the connecting member 40b to engage with the peripheral portion of the mounting hole 55b at the same time that the connecting member 40b is inserted into the mounting hole 55b.

In this way, according to the blocking device 10 of the first representative embodiment, the blocking member 20 may be fixed in position by the connecting members 40a and 40b that extend through the mounting holes 55a and 55b formed in the reinforcement panel 53 or by the fixing member 30 that is connected to the blocking device 10 via the connecting members 40a and 40b. Therefore, the blocking member 20 may be reliably and fixedly mounted within the first space 54a. As a result, even in the event that a liquid coating material for electro-deposition (known as a ED liquid) has surged into the space 54 of the hollow structure 50, the blocking member 20 may be effectively prevented from failure due to the pressure of the coating material.

Further, as shown in FIG. 1, ribs 47a and 47c are integrally formed with the first holder plate 21 and extend substantially perpendicular to the first holder plate 21. Similarly, ribs 47b and 47d are integrally formed with the second holder plate 22 and extend substantially perpendicular to the second holder plate 22. The ribs 47a to 47d may serve as support legs for maintaining the blocking member 20 in an upright position within the first space 54a. Therefore, with the aid of the ribs 47a to 47d the blocking member 20 may be further effectively prevented from failure due to the pressure of the coating material.

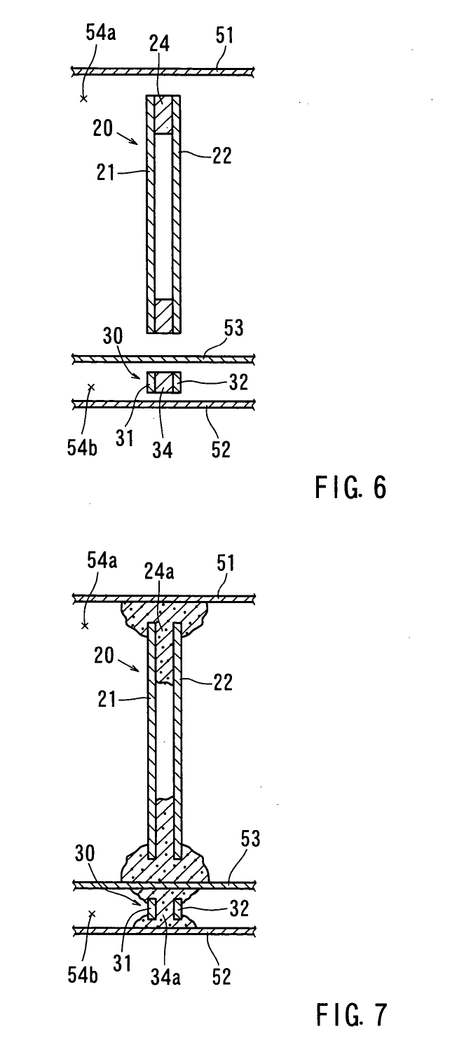

In case that the hollow structure 50 is a hollow panel of an automobile body (e.g., a pillar, a rocker panel, a roof side panel, or a wheel housing), the internal surface and the external surface of the automobile body or the hollow panel may be coated with an electro-deposition coating. The automobile body may then be conveyed into a heating furnace where the coatings are baked. Therefore, the expandable members 24 and 34 of the blocking device 10 shown in FIG. 6 may expand to respectively form foam members 24a and 34b, as shown in FIG. 7, as a result of the external heat applied to the automobile body during the baking process of the coatings. As shown in FIG. 7, the expansion of the expandable members 24 and 34 develops along the corresponding wall surfaces of the hollow structure 50. The resulting foam members 24a and 34a may closely contact with the corresponding wall surfaces of the hollow structure 50. Consequently, the first space 54a and the second space 54b may be respectively blocked by the foam members 24a and 34a without allowing substantial clearances to form between the blocking member 20 and the corresponding surrounding wall surfaces and between the fixing member 30 and the corresponding surrounding wall surfaces. With the first and second spaces 54a and 54b blocked by the foam members 24a and 34b of the blocking member 20 and the fixing member 30, vibration damping, sound insulating properties, and structural reinforcement may be provided to the hollow structure 50.

As described above, according to the blocking device 10 of this representative embodiment, the blocking member 20 may be reliably prevented from failing within the first space 54a. Therefore, it is possible to prevent the expandable member 24 from expanding in an unintended direction within the first space 54a. As a result, the formed foam member 24a can reliably block the first space 54a.

In addition, the fixing member 30 used for fixing the blocking member 20 in a correct position also can serve to block an interior space. As a result, in the case that the space 54 of the hollow structure 50 is divided into a first and second space 54a and 54b by a reinforcement panel 53 or a partition extending along the longitudinal direction of the hollow structure 54, the fixing member 30 can block the second space 54b while the blocking member 20 blocks the first space 54a. In this way, the two separate spaces within the hollow structure 54 can be simultaneously blocked.

In the above representative embodiment, the connecting members 40a and 40b of the fixing member 30 have respective latching holes 41a and 41b through which the clips 43a and 43b extend in order to prevent the connecting members 40a and 40b from being inadvertently removed from the blocking member 20. However, the present invention may not be limited to this construction. For example, the connecting members 40a and 40b may be formed with other clips or engaging members that may prevent them from being removed from the expandable member 24 of the blocking member 20. A variety of examples of such modifications (modifications 1 to 3) will be hereinafter described. For the purpose of illustration, these modifications will be described in relation to only one connecting member. Of course, the same modification may be applied to a number of connecting members.

(Modification 1)

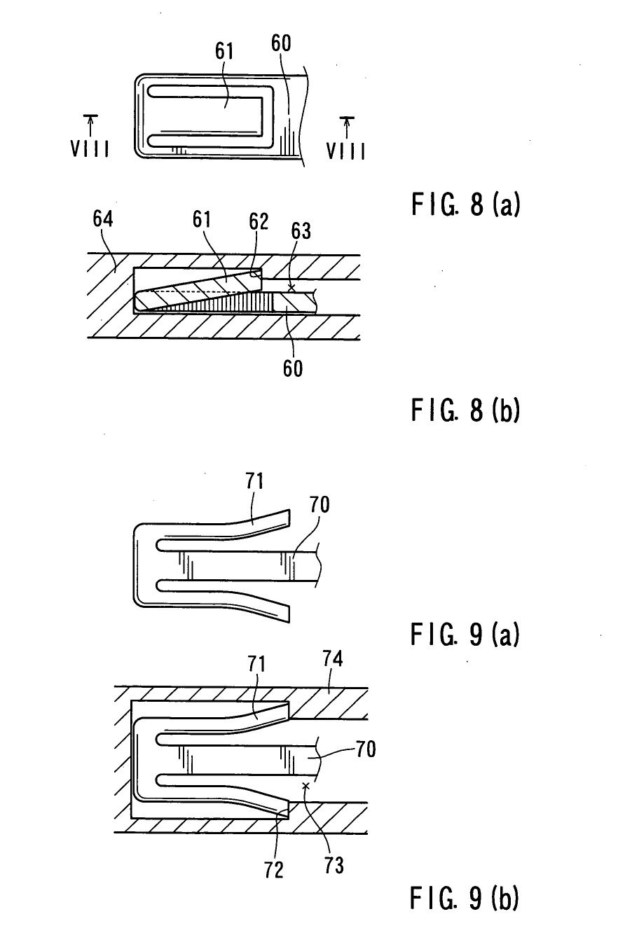

According to Modification 1 shown in FIGS. 8(a) and 8(b), a clip or a resilient engaging member 61 is integrally formed with an end portion of a connecting member 60. As shown in FIG. 8(a), the engaging member 61 is formed by cutting out a substantially U-shaped slot in the end portion of the connecting member 61 and bending a portion surrounded by the U-shaped slot upward, as viewed in FIG. 8(b). An insertion hole 63, formed in an expandable member 64 of the blocking member 20, has a stepped portion 62 formed on the upper wall as viewed in FIG. 8(b) of the insertion hole 63. This allows the engaging member 61 to engage with the stepped portion 62 when the connecting member 60 has been inserted into the insertion hole 63. With this arrangement, the fixing member 30 can be very easily connected to the blocking member 20 by the connecting member 60. In addition, it is possible to easily prevent the connecting member 61 from being inadvertently removed from the insertion hole 63.

(Modification 2)

According to Modification 2 shown in FIGS. 9(a) and 9(b), a pair of resilient engaging members 71 that may form a clip together are formed integrally with an end portion of a connecting member 70. As shown in FIG. 9(a), the engaging members 71 extend from the terminal end of the connecting member 70 in a turn-back manner. An insertion hole 73 formed in an expandable member 74 of the blocking member 20 has stepped portions 72 formed on the upper and lower walls, as viewed in FIG. 9(b), of the insertion hole 73. This allows the engaging members 71 to engage with the stepped portions 72 when the connecting member 70 has been inserted into the insertion hole 73. Also with this arrangement, the fixing member 30 can be very easily connected to the blocking member 20 by the connecting member 70. In addition, it is possible to easily prevent the connecting member 71 from being inadvertently removed from the insertion hole 73.

(Modification 3)

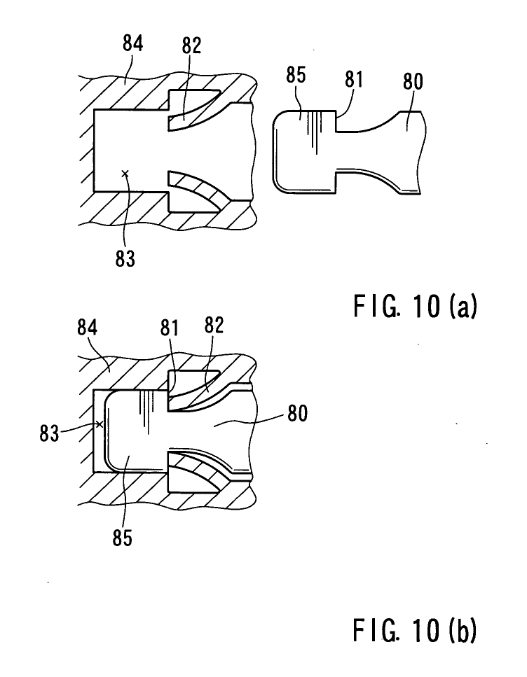

According to Modification 3 shown in FIGS. 10(a) and 10(b), an end portion of a connecting member 80 is configured as a clip or an engaging member 85 having a rounded front end. Stepped portions 81 are formed on the rear side of the engaging member 85. Correspondingly, resilient engaging members 82 are integrally formed with the upper and lower walls, as viewed in FIGS. 10(a) and 10(b), of an insertion hole 83 that is formed in an expandable member 84 of the blocking member 20. Therefore, the engaging members 82 may engage with the stepped portions 81 of the engaging member 85 when the connecting member 80 has been inserted into the insertion hole 83 as shown in FIG. 10(b). Also with this arrangement, the fixing member 30 can be very easily connected to the blocking member 20 by the connecting member 80. In addition, it is possible to easily prevent the connecting member 81 from being inadvertently removed from the insertion hole 83.

(Other Possible Modifications)

In the above representative embodiment, a pair of holder plates 31 and 32 and the expandable member 34 constitute the fixing member 30. However, the fixing member 30 may not be limited to this construction. Since a primary function of the fixing member 30 may be to fix the blocking member 20 in position within the space of the hollow structure, the fixing member 30 may be constructed so as to be dedicated to this specific function. For example, the fixing member 30 may consist entirely of a single element molded from thermoplastic resin, such as 66-nylon.

In addition, although the above representative embodiment has been described in relation to the hollow structure 50 that may be a pillar, a rocker panel, a roof side panel, or a wheel housing of an automobile body for example, the present invention may also be applied to any other hollow structures in addition to those of automobiles. For example, the present invention may be applied to the hollow structures of houses, buildings, boats, and ships.

1. A blocking device for mounting within a space defined within a hollow structure in which the hollow structure has a wall portion extending into the space, the blocking device comprising:
a blocking member arranged and constructed to be disposed on one side of the wall portion within the space, comprising:
an expandable member, and
a holder for holding the expandable member,
wherein the expandable member is expandable by heat to form a foam member; and
a fixing device comprising:
a fixing member arranged and constructed to be disposed on the other side of the wall portion within the space, and
a connecting member arranged and constructed to extend through an insertion hole formed in the wall portion to connect the blocking member to the fixing member.

2. The blocking device as in claim 1,
wherein the fixing device comprises a plurality of the connecting members; and
wherein a plurality of the insertion holes are formed in the wall portion corresponding to the plurality of the connecting members.

3. The blocking device as in claim 1,
wherein the expandable member has a first side and a second side opposite to each other; and
wherein the holder comprises a first holder plate and a second holder plate respectively disposed on the first side and the second side of the expandable member; and
wherein the first holder plate has an engaging member; and
wherein the second holder plate has an engaging hole positioned to correspond to the engaging member; and
wherein the connecting member has a latching hole, so that the first engaging member engages with the engaging hole via the latching hole.

4. The blocking device as in claim 3,
wherein the first holder plate has a plurality of the engaging members; and
wherein the second holder plate has a plurality of the engaging holes corresponding to the plurality of the engaging members; and
wherein the fixing device has a plurality of the connecting members each having the latching hole.

5. The blocking device as in claim 3, further comprising an engaging claw integrally formed with a circumferential edge of the engaging hole in order to inhibit the removal of the engaging member from the engaging hole.

6. The blocking device as in claim 1,
wherein the connecting member has an engaging member disposed on an end portion of the connecting member; and
wherein the blocking member has an engaging hole for engagement with the engaging member; and
wherein the engaging hole has an engaging portion formed on an inner wall of the engaging hole and engageable with the engaging member in order to inhibit the removal of the engaging member from the engaging hole.

7. The blocking device as in claim 6,
wherein the fixing device comprises a plurality of the connecting members respectively having the engaging members; and
wherein the blocking member has a plurality of the engaging holes corresponding to the engaging members.

8. The blocking device as in claim 6,
wherein the engaging member is formed integrally with the holder; and
wherein the connecting member is formed integrally with the fixing member.

9. The blocking device as in claim 1,
wherein the holder has at least one rib for maintaining the blocking member in an upright position.

10. The blocking device as in claim 1,
wherein the fixing member comprises a second expandable member and a second holder for holding the second expandable member.
wherein the expandable member is expandable by heat to form a second foam member.

11. A blocking device for mounting within a space defined within a hollow structure, the hollow structure having a wall portion extending into the space, comprising:
a blocking member arranged and constructed to be disposed within the space of the hollow structure;
wherein the blocking member comprises an expandable member expandable by heat to form a foam member; and
a fixing member arranged and constructed to be fixed to the wall portion of the hollow structure; and
a connecting member arranged and constructed to connect the fixing member to the blocking member and to extend through an insertion hole formed in the wall portion.

12. The blocking device as in claim 11,
wherein the connecting member is formed integrally with the fixing member.

13. The blocking device as in claim 11,
wherein the fixing member comprises a second expandable member expandable by heat to form a second foam member.

14. The blocking device as in claim 13,
wherein the wall portion of the hollow structure is a partition wall separating the space into a first space and a second space; and
wherein the blocking member and the fixing member are arranged and constructed to be respectively disposed within the first space and the second space and to block the first space and the second space when expanded.

15. The blocking device as in claim 11,
wherein the connecting member comprises an engaging member arranged and constructed to engage with the blocking member, so that the blocking member is prevented from being accidentally removed from the fixing member after engagement by the engaging member.
